(19) United States
(12) Patent Application Publication
(10) Pub. No.: US 2025/0242084 A1
(43) Pub. Date: Jul. 31, 2025

(54) REFILLABLE TISSUE ENGINEERED SCAFFOLDS

(71) Applicant: Board of Regents of the University of Nebraska, Lincoln, NE (US)

(72) Inventors: BIN DUAN, Omaha, NE (US); TAMMY KIELIAN, LaVista, NE (US); LEE KORSHOJ, Omaha, NE (US); WEN SHI, Papillion, NE (US)

(21) Appl. No.: 18/699,799

(22) PCT Filed: Oct. 20, 2022
(86) PCT No.: PCT/US22/47235

§ 371 Date: Apr. 9, 2024

Related U.S. Application Data
(60) Provisional application No. 63/270,259, filed on Oct. 21, 2021.

(51) Int. Cl. A61L 27/54; A61L 27/20; A61L 27/34; A61L 27/52; C08L 5/04

(52) U.S. Cl. CPC A61L 27/54; A61L 27/20; A61L 27/34; A61L 27/52; C08L 5/04; A61L 2300/406; A61L 2400/06; A61L 2420/06

(57) ABSTRACT

Compositions and methods for delivering therapeutics to a subject are provided.

This application claims priority under 35 U.S.C. § 119 (e) to U.S. Provisional Patent Application No. 63/270,259, filed Oct. 21, 2021. The foregoing application is incorporated by reference herein.

### FIELD OF THE INVENTION

This application relates to the fields of tissue engineering scaffolds and therapeutic delivery. More specifically, this invention provides compositions and methods for delivering therapeutics, particularly for inhibiting infections associated with implant surgeries, craniotomies, and other related procedures.

### BACKGROUND OF THE INVENTION

Several publications and patent documents are cited throughout the specification in order to describe the state of the art to which this invention pertains. Each of these citations is incorporated herein by reference as though set forth in full.

Battlefield case fatality rates (CFR) have declined steadily throughout the twentieth century, from 19.1% among all wounded in WWII, to 15.8% in Vietnam, and 9.4% in Operation Iraqi Freedom and Operation Enduring Freedom (OIF and OEF, respectively) (Blyth, et al. (2015) J. Trauma Acute Care Surg. 79 (4 Suppl 2): S227-235; Holcomb, et al. (2006) J. Trauma. 60 (2): 397-401). The frequency of head wounds during OIF/OEF was dramatically increased compared to earlier conflicts, with most injuries caused by explosives (79%), of which 38% were improvised explosive devices (IEDs) (Owens, et al. (2008) J. Trauma. 64 (2): 295-299). Traumatic brain injury (TBI) has been estimated to account for up to one-third of combat-related injuries on today's battlefield (Pavlicevic, et al. (2017) J. Craniomaxillofac. Surg., 45 (2): 312-318; Meyer, et al. (2008) J. Trauma Nurs., 15 (4): 185-189). Indeed, during OIF and OEF, at least 8,089 TBIs were recorded from IEDs, many of which required life-saving decompressive craniectomy (Ling, et al. (2010) Annu. Rev. Med., 61:457-468). Decompressive craniectomy refers to the surgical removal of a portion of the skull following TBI (or other head-related injuries such as stroke or cranial bleeding) to control subsequent brain edema and prevent death (Stiver, S. I. (2009) Neurosurg. Focus., 26 (6): E7). Upon removal, the bone fragment (or bone flap) is typically cryopreserved until future replacement once brain edema has subsided. A surge in decompressive craniectomies has been observed in the last decade, due to reported benefits in the setting of failed medical treatment for intracranial hypertension secondary to TBI and stroke (Hutchinson, et al. (2006) Acta Neurochir. Suppl., 96:17-20; Vahedi, et al. (2007) Stroke 38 (9): 2506-2517).

A craniotomy refers to the temporary removal of a skull fragment to access the brain, such as during tumor resection or epilepsy treatment, which is replaced intra-operatively. The incidence of infection after craniotomy/craniectomy ranges from 0.8-12% in the modern surgical era, with approximately half attributed to Staphylococcus aureus (S. aureus), which forms a biofilm on the native bone (McClelland, et al. (2007) Clin. Infect. Dis., 45 (1): 55-59; Chiang, et al. (2011) J. Neurosurg., 114 (6): 1746-1754). In patients with cranial defects resulting from combat-related injuries, infection can be assumed, with S. aureus among the more prevalent pathogens leading to biofilm formation (Stephens, et al. (2010) Neurosurg. Focus, 28 (5): E3). Several factors have been identified that increase the risk for infectious complications after craniotomy, including the presence of another infection, number of operations, cerebrospinal fluid (CSF) leakage, extent of CSF drainage, and venous sinus entry. It has been proposed that CSF leakage promotes the retrograde movement of bacteria, resulting in intracranial infection, which increases intracranial pressure and further CSF leakage, perpetuating a vicious cycle (Fang, et al. (2017) Am. J. Infect. Control 45 (11): e123-e134).

Historically, the standard-of-care for managing bone flap infection following craniotomy has been intra-operative debridement of the affected tissue and bone flap removal, whereupon after a prolonged course of antibiotic treatment, a cranioplasty is performed. However, some studies have reported successful regimens for salvaging infected bone flaps, which include aggressive debridement of the surgical site, scrubbing and soaking the bone flap in povidone-iodine or other antiseptics, and in some instances indwelling antibiotic irrigations systems have been used to bathe the bone flap in antibiotics upon reinsertion (Widdel, et al. (2009) J. Neurosurg. Pediatr., 4 (4): 378-382; Auguste, et al. (2006) J. Neurosurg., 105 (4): 640-644; Bruce, et al. (2003) J. Neurosurg., 98 (6): 1203-1207; Wallace, et al. (2018) Neurosurg. Rev., 41 (4): 1071-1077). In all of these studies, patients received intravenous (i.v.) antibiotics on average for one week followed by a longer duration of oral antibiotics (i.e., 2-3 months) (Widdel, et al. (2009) J. Neurosurg. Pediatr., 4 (4): 378-382; Auguste, et al. (2006) J. Neurosurg., 105 (4): 640-644; Bruce, et al. (2003) J. Neurosurg., 98 (6): 1203-1207; Wallace, et al. (2018) Neurosurg. Rev., 41 (4): 1071-1077). Although these studies have shown good efficacy in preventing infection recurrence, they report small sample sizes (i.e., <14 subjects) and have not yet been adapted into mainstream clinical practice.

In instances where the infected bone flap cannot be salvaged, patients are typically subjected to at least two additional surgeries. The first is to remove the infected bone, and after a variable period of antibiotic treatment that can last for weeks to months, a second surgery occurs to seal the cranial cavity with an alloplastic prosthesis or autologous bone graft (Baumeister, et al. (2008) Plast. Reconstr. Surg., 122 (6): 195e-208e). Prolonged absence of the skull flap during antibiotic treatment can lead to “syndrome of the trephined” in approximately 13% of patients, which can include headache, seizures, mood imbalances, and behavioral disturbances (Honeybul, et al. (2011) J. Neurotrauma 28 (6): 929-935). Treatment of trephine syndrome is replacement of the original bone flap or synthetic device (Sinclair, et al. (2010) Radiographics 30 (2): 461-482; Yang, et al. (2008) Acta Neurochir 150 (12): 1241-1248). However, replacement cannot occur until convincing evidence exists that any residual infection has been eliminated, and some patients experience lingering cognitive impairment. Although less common, patients with craniotomy/craniectomy infections can experience chronic seizures and focal neurological deficits (Bhaskar, et al. (2014) World Neurosurg., 82 (3-4): e531-e534). Despite the extensive steps taken both pre- and post-operatively to prevent infectious complications following craniotomy, including surgical scrub techniques, peri-operative antibiotics, post-operative wound care, and discontinuation of wound healing retardant medications, infections still occur and some require the bone flap to be discarded. Improved methods for preventing microbial infections after a craniotomy/craniectomy are clearly needed.

### SUMMARY OF THE INVENTION

In accordance with the instant invention, methods of delivering a therapeutic to a subject in need thereof are provided. Methods of treating, inhibiting, and/or preventing a microbial or bacterial infection (including biofilms) in a subject in need thereof are also provided. In certain embodiments, the methods of the instant invention comprise a) administering or implanting a functionalized hydrogel to a subject, wherein the functionalized hydrogel comprises a hydrogel covalently attached directly or via a linker to a reactant of a click reaction, and b) administering a functionalized therapeutic and/or functionalized vesicle comprising or encompassing a therapeutic to the subject, wherein the functionalized therapeutic comprises a therapeutic covalently attached directly or via a linker to the counterpart reactant for the click reaction, and wherein the functionalized vehicle comprises a vehicle covalently attached directly or via a linker to the counterpart reactant for the click reaction. In certain embodiments, the functionalized hydrogel comprises an azide, and the functionalized therapeutic and/or functionalized vesicle comprises an alkyne group (e.g., DBCO). In certain embodiments, the functionalized hydrogel comprises an alkyne group (e.g., DBCO), and the functionalized therapeutic and/or functionalized vesicle comprises an azide. In certain embodiments, the functionalized hydrogel coats and/or is contained within a structure or scaffold. In certain embodiments, step b) comprises systemic administration such as by intravenous and/or intraperitoneal administration. In certain embodiments, step b) is performed more than once. For example, step b) can be performed as needed by the subject for therapeutic effect. In certain embodiments, the functionalized hydrogel comprises alginate. In certain embodiments, the therapeutic is an antimicrobial or antibiotic. In certain embodiments, the functionalized hydrogel comprises alginate covalently attached to dibenzocyclooctyne (DBCO) via a linker, the functionalized therapeutic is an antibiotic (e.g., vicomycin) covalently attached directly or via a linker to an azide group, and the functionalized vesicle is a liposome encapsulating an antibiotic (e.g., daptomycin) covalently attached directly or via a linker to an azide group. In certain embodiments, the functionalized hydrogel comprises alginate covalently attached to an azide via a linker, the functionalized therapeutic is an antibiotic (e.g., vancomycin) covalently attached directly or via a linker to dibenzocyclooctyne (DBCO), and the functionalized vesicle is a liposome encapsulating an antibiotic (e.g., daptomycin) covalently attached directly or via a linker to dibenzocyclooctyne (DBCO). In certain embodiments, the hydrogel is administered or implanted at a site of a prosthetic or surgical implant. In certain embodiments, the hydrogel is administered or implanted to bone or a bone flap (e.g., cranial bone flap). In certain embodiments, the hydrogel is administered or implanted at a site of infection (e.g., an infection at a prosthetic or surgical implant).

### BRIEF DESCRIPTION OF THE DRAWINGS

FIG. 1A provides a schematic of the synthesis of DBCO-modified alginate. FIG. 1B provides a schematic of the synthesis of Cy5 labeled DBCO-modified alginate by a click reaction. FIG. 1C provides images of alginate and alginate-DBCO hydrogels incubated with a sulfo-Cy5-N₃ solution followed by washing with phosphate buffered saline (PBS). FIG. 1D provides a graph of the fluorescence intensity of Cy5 labeled alginate hydrogels and Cy5 labeled DBCO-modified alginate hydrogels measured daily for five days by a plate reader while incubated in water. FIG. 1E provides nuclear magnetic resonance (NMR) spectra of alginate and alginate-DBCO.

FIG. 2A provides a schematic of the structure of daptomycin. FIG. 2B provides a schematic of the structure of 1,1′-dioctadecyl-3,3,3′,3′-tetramethylindodicarbocyanine (DiD). FIG. 2C provides a schematic of the structure of DSPE-PEG-N₃, wherein n defines the length of the polyethylene glycol (PEG).

FIG. 3A provides a graph of the size of liposomes as determined by dynamic light scattering. FIG. 3B provides images of alginate or alginate-DBCO hydrogels reacted with daptomycin-loaded mPEG liposomes or daptomycin-loaded azide liposomes. Hydrogels were washed in saline for 20 minutes prior to imaging. FIG. 3C provides a graph of the fluorescence intensity of the hydrogels of FIG. 3B. ****p<0.0001. FIG. 3D provides a graph of daptomycin release from liposomes.

FIG. 4A provides images of plates spread with methicillin-resistant Staphylococcus aureus (MRSA) and containing alginate and alginate-DBCO hydrogels after incubation with DSPE-N₃ liposomes or DSPE-mPEG liposome. Images were taken after an overnight incubation. FIG. 4B provides a graph of the zone of inhibition. * p<0.01, ***p<0.001.

FIG. 5A provides IVIS® images collected 24 hours after intravenous dosing of DSPE-N₃ liposome loaded with DiD dye and daptomycin into mice with alginate or alginate-DBCO hydrogels placed above the bone flap following sterile craniotomy seven days prior to dosing. FIG. 5B provides a graph of the total radiance within the marked regions of interest from FIG. 5A. ***p<0.001. FIG. 5C provides IVIS® images of extracted hydrogels from female mice at day 3 post-dosing.

FIG. 6A provides a schematic of the synthesis of azide modified alginate hydrogel. FIG. 6B provides a schematic of click reaction between DBCO modified Cy5 and azide modified alginate. FIG. 6C provides images of alginate and alginate-N₃ hydrogels (n=4) incubated with sulfo-Cy5-DBCO solution followed by washing with PBS. Positive control of sulfo-Cy5-DBCO solution also provided (top).

FIG. 7A provides IVIS® images after intravenous dosing of Cy5-N₃ dye into mice with alginate or alginate-DBCO hydrogels. Hydrogels with incorporated 3D bioprinted hydroxyapatite scaffold were placed above the bone flap following sterile craniotomy seven days prior to dosing. FIG. 7B provides IVIS® images of extracted hydrogels and scaffolds from mice at day 5 post-dosing. FIG. 7C provides a graph of the total radiance from IVIS® images of extracted hydrogels. **p<0.01.

FIG. 8A provides a schematic of the structure of the antibiotic vancomycin. FIG. 8B provides a schematic of the synthesis of azide-modified vancomycin using an imine based modification approach. FIG. 8C provides a schematic of the synthesis of azide-modified vancomycin using an anhydride based modification approach.

FIG. 9 provides a schematic of the synthesis of DBCO vancomycin prodrug.

### DETAILED DESCRIPTION OF THE INVENTION

In accordance with the instant invention, implantable, hydrogel-based scaffolds are provided that can serve as a refillable system to administer therapeutics. For example, the compositions and methods of the instant invention can be used to directly release therapeutics (e.g., antibiotics) and/or sequester systemically administrated therapeutics (e.g., antibiotics) to prevent infection, mitigate biofilm formation, and/or manage infectious complications after surgery such as with prosthetic and/or surgical implants (e.g., a craniotomy/craniectomy procedure). The scaffolds of the instant invention may be synthesized using biocompatible materials. Modified and/or encapsulated therapeutics (e.g., antibiotics) may be administered to refill the scaffolds, thereby decreasing the administration frequency and providing sustained release of the therapeutics.

The compositions and methods of the instant invention can be used to prevent infection and recurrence and/or treat or inhibit an infection. With infectious complications minimized, the compositions and methods of the instant invention improve the recovery time and limit adverse outcomes by frequent administration of antibiotics, thereby saving significant time and resources spent on medical procedures and care, as well as improving clinical outcomes for patients.

Briefly, the structures or scaffolds of the instant invention can be used as implantable, refillable, and stimuli-responsive for localized therapeutic release at the target site. For example, the structures of scaffolds can be implanted into a cranial cavity after a craniotomy/craniectomy surgery, particularly when the native bone cannot be salvaged. The compositions and methods of the instant invention will allow for the inhibition or prevention of cranial bone flap infection that can lead to major complications, including morbidity and mortality. The compositions and methods of the instant invention also allow for the inhibition or prevention of the recurrence of infection after the surgical removal of an infected bone flap. The structure or scaffold are generally hydrogel based scaffolds (e.g., alginate) with modification to click react with modified/encapsulated therapeutics such as antibiotics (e.g., daptomycin or vancomycin). Typically, the hydrogel scaffold is implanted into subject at the desired location (e.g., the cranial cavity) and then the systemic administration (e.g., either i.v. or i.p.) of the modified/encapsulated therapeutic (e.g., antibiotics) allows for the click reaction and loading of the therapeutic onto the hydrogel. The “clicked” therapeutic (e.g., antibiotics) will be slowly released, thereby providing sustained treatment to the area (e.g., for in situ mitigation of biofilm and/or inhibition of bacterial growth). The therapeutic (e.g., antibiotics) can be administrated more than once to effectively refill the hydrogel scaffold to achieve long-term effects.

In accordance with the instant invention, functionalized hydrogels are provided. In certain embodiments, the hydrogel is functionalized with a compound comprising a reactant of a click chemistry reaction. In certain embodiments, the click chemistry reaction is an azide-alkyne cycloaddition (e.g., Pickens et al., Bioconjugate Chem. (2018) 29 (3): 686-701). In certain embodiments, the hydrogel is functionalized with an azide group (N₃). In certain embodiments, the hydrogel is functionalized with an alkyne group (carbon-carbon triple bond). In certain embodiments, the reactant of the click chemistry reaction is covalently attached to the hydrogel directly or via a linker. In certain embodiments, a compound comprising the reactant of the click chemistry reaction is covalently attached to the hydrogel directly or via a linker. Compositions comprising the hydrogel, optionally with a structure or scaffold as described herein, and at least one pharmaceutically acceptable carrier are also encompassed by the instant invention.

Generally, a linker is a chemical moiety comprising a covalent bond or a chain of atoms that covalently attaches the two compounds. The linker can be linked to any synthetically feasible position of the compounds, particularly without significantly affecting the activity of the compounds, if applicable. Exemplary linkers may comprise at least one optionally substituted; saturated or unsaturated; linear, branched or cyclic aliphatic group, an alkyl group, or an optionally substituted aryl group. In a particular embodiment, the linker is an optionally substituted aliphatic or alkyl group. The aliphatic or alkyl group may be unsaturated or saturated and may be substituted with at least one heteroatom (e.g., O, N, or S). In a particular embodiment, the alkyl or aliphatic group comprises about 1 to about 30 carbons (e.g., in the main chain of the alkyl or aliphatic group), which may be substituted with at least one heteroatom (e.g., O, N, or S). In a particular embodiment, the linker comprises polyethylene glycol (e.g., about 1 to about 10, about 1 to about 9, about 1 to about 8, about 1 to about 7, about 1 to about 6, about 1 to about 5, about 1 to about 4, about 1 to about 3, about 1 to about 2 PEG units, or 1 PEG unit; or about 2 to about 8 or about 3 to about 6 PEG units).

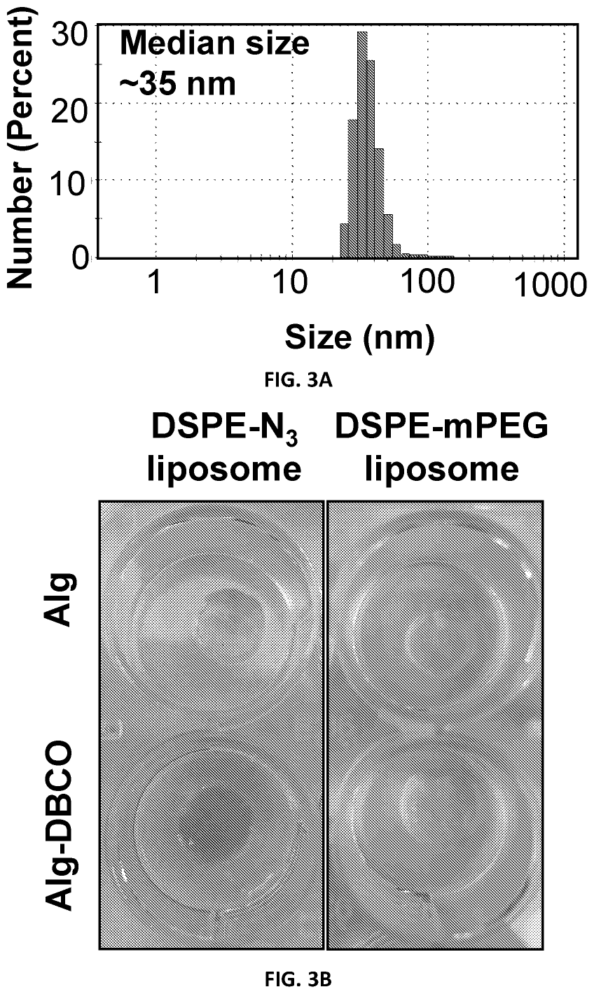

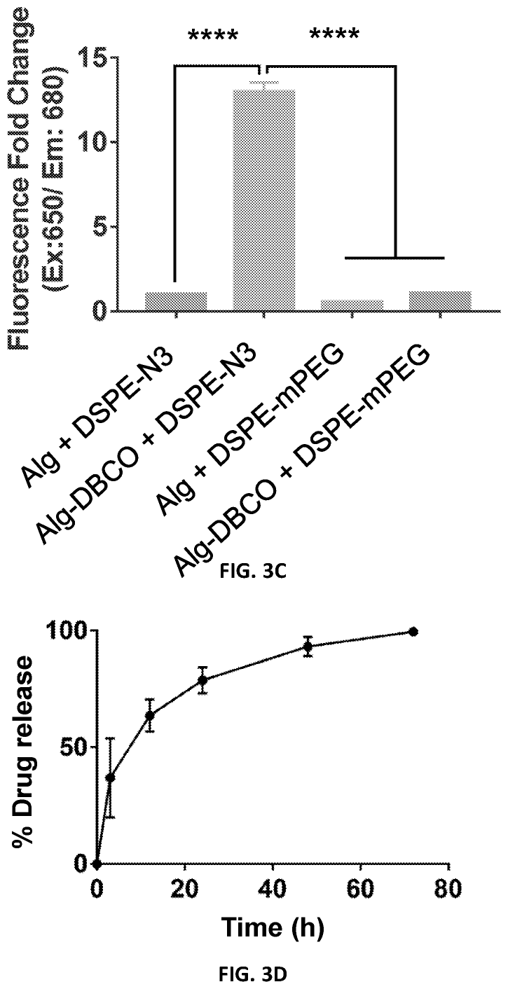

In certain embodiments, the hydrogel is covalently attached directly or via a linker to a compound comprising an alkyne group. Examples of compounds comprising an alkyne group are provided in Pickens et al. (Bioconjugate Chem. (2018) 29 (3): 686-701 (e.g., FIG. 3); incorporated by reference herein). Examples of compounds comprising alkyne include, without limitation: DBCO, DIBAC, DIFO, DIBO, BCN, BARAC, and derivatives thereof. In certain embodiments, the hydrogel is covalently attached directly or via a linker to a compound comprising dibenzocyclooctyne (DBCO). In certain embodiments, the hydrogel is covalently attached via a linker to dibenzocyclooctyne (DBCO). In certain embodiments, the functionalized hydrogel comprises alginate covalently attached via a linker to dibenzocyclooctyne (DBCO). In certain embodiments, the functionalized hydrogel is alginate, wherein the compound comprising an alkyne group (e.g., DBCO) is covalently attached directly or via a linker to a —COOH group of alginate. In certain embodiments, the linker comprises polyethylene glycol.

In certain embodiments, the hydrogel is covalently attached directly or via a linker to a compound comprising an azide group. Examples of compounds comprising an azide group are provided in Pickens et al. (Bioconjugate Chem. (2018) 29 (3): 686-701 (e.g., FIG. 3); incorporated by reference herein). In certain embodiments, the hydrogel is covalently attached directly or via a linker to an azide group. In certain embodiments, the hydrogel is covalently attached via a linker to an azide group. In certain embodiments, the functionalized hydrogel comprises alginate covalently attached via a linker to an azide group. In certain embodiments, the functionalized hydrogel is alginate, wherein the compound comprising an azide is covalently attached directly or via a linker to a —COOH group of alginate. In certain embodiments, the linker comprises polyethylene glycol.

In certain embodiments, the hydrogel is a macromolecular polymer gel including a network. In certain embodiments, the hydrogel is a polymer matrix able to retain water in a swollen state. In certain embodiments, the hydrogel is crosslinked. Examples of hydrogels include, without limitation, one or more of: gelatin, alginate, chitosan, collagen, silk, fibrin, agarose, chondroitin, elastin, starch, pectin, cellulose, methylcellulose, polyethylene glycol (PEG), polyvinyl alcohol (PVA), sodium polyacrylate, polyacrylamide, starch-acrylonitrile co-polymers, a proteoglycan, elastin, and/or a glycosaminoglycan (e.g., hyaluronic acid, heparin, chondroitin sulfate, or keratan sulfate), other natural or synthetic hydrogels, and derivatives thereof (e.g., del Valle et al., Gels (2017) 3:27). In certain embodiments, the hydrogel is selected from the group consisting of alginate, chitosan, hyaluronic acid, gelatin, silk, fibrin, collagen, elastin, cellulose, agarose, chondroitin, PEG, PVA, and polyacrylamide. In certain embodiments, the hydrogel is biocompatible. In certain embodiments, the hydrogel is biodegradable. In certain embodiments, the hydrogel is non-biodegradable. In certain embodiments, the hydrogel comprises alginate.

Crosslinking may be done using a variety of techniques including thermal crosslinking, ionic crosslinking, chemical crosslinking, and photo-crosslinking. For example, the 3D printed scaffolds of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent.

In certain embodiments, the hydrogel coats and/or is contained within a structure or scaffold. For example, the structure may be a natural or biologic substrate such as bone or cartilage (e.g., from the subject to be treated (autologous) or from another (including a cadaver)). In certain embodiments, the hydrogel coats a bone flap (e.g., for a craniotomy/craniectomy procedure). In certain embodiments, the structure is a prosthetic and/or surgical implant. In certain embodiments, the scaffold is a 3D printed scaffold. In certain embodiments, the structure is a biomimetic (e.g., of the skull or bone flap). In certain embodiments, the structure or scaffold comprises hydroxyapatite.

The term “coat” refers to a layer of a substance/material on the surface of a structure. Coatings may, but need not, also impregnate the structure. Further, while a coating may cover 100% of the structure, a coating may also cover less than 100% of the surface of the structure (e.g., at least about 75%, at least about 80%, at least about 85%, at least about 90%, at least about 95%, at least about 98%, or more of the surface may be coated).

The artificial or synthetic scaffolds of the instant invention can be synthesized by any means. For example, the scaffolds of the instant invention can be 3D printed. The 3D printed scaffolds can be printed to the desired size and/or shape. The 3D printed scaffolds can also be trimmed or cut after production to the desired size and/or shape. For example, the 3D printed scaffold may be printed as a patient-specific product or the 3D printed scaffold may be produced as an off-the-shelf product which is later trimmed and/or cut for a specific patient's needs. In certain embodiments, the 3D printed scaffold is designed to fit and/or be implanted into a cavity such as a bone or cranial cavity, such as after a craniotomy/craniectomy surgery. For example, the shape of the 3D printed scaffold may be determined by scanning the cavity or cranial cavity (e.g., by a computed tomography (CT) scan). Methods of 3D printing implantable scaffolds are known in the art (see, e.g., Nowicki, et al. (2016) Nanotechnology 27:414001; Rutz, et al. (2015) Advanced Materials 27:1607-1614; Suri, et al. (2011) Biomed Microdevices 13:983-993; Shim, et al. (2016) Biofabrication 8:014102; Duan, et al. (2010) Acta Biomater., 6 (12): 4495-4505; Duan, et al. (2010) J. R. Soc. Interface., 7 Suppl 5: S615-629, each of which is incorporated by reference herein). The 3D printed scaffolds may be printed with a high-resolution 3D printer (e.g., a 3D-Bioplotter® Manufacturer Series (envisontec, Dearborn, MI)). The 3D printed scaffolds of the instant invention may be crosslinked (e.g., to enhance their stability).

After synthesis, the scaffolds (e.g., 3D printed scaffolds) may be washed or rinsed in water and/or a desired carrier or buffer (e.g., a pharmaceutically or biologically acceptable carrier). The scaffolds may also be stored in a cold solution, lyophilized and/or freeze-dried (e.g., before the addition of cells). The scaffolds of the instant invention may also be sterilized (e.g., before the addition of cells). For example, the scaffolds can be sterilized using various methods (e.g., by treating with ethylene oxide gas, gamma irradiation, ultraviolet radiation, or 70% ethanol).

The scaffolds of the instant invention can be of any desired size. In certain embodiments, the scaffold has a thickness of less than about 15 mm, less than about 10 mm, less than about 8 mm, less than about 5 mm, or less than about 3 mm. In a particular embodiment, the scaffolds have a thickness of about 0.5 mm to about 10 mm, about 0.5 mm to about 5 mm, about 0.5 mm to about 3 mm, or about 1 mm to about 3 mm.

In certain embodiments, the scaffold (e.g., 3D printed scaffold) comprises one or more biocompatible materials. In certain embodiments, the 3D printed scaffold comprises one or more FDA approved biocompatible materials. The biocompatible material may be biodegradable or non-biodegradable. In certain embodiments, the scaffold comprises a single biocompatible material. In certain embodiments, the scaffold comprises more than one biocompatible material. For example, the scaffold may comprise layers of different biocompatible materials. The scaffold may comprise interlaced or interwoven strands of one or more biocompatible materials. For example, a first biocompatible material may be printed in spaced lines and/or frame, then a second biocompatible material may be printed in between the lines and/or frame of the first biocompatible material. In certain embodiments, one of the biocompatible materials is hydrophobic and the other biocompatible material is hydrophilic. In certain embodiments, the 3D printed scaffold is printed in a layer-by-layer manner, optionally wherein the scaffold is rotated 90° at each successive layer to create a grid pattern in the scaffold.

The biocompatible materials of the instant invention include, without limitation: ceramics (e.g., bioceramics), glasses, polymers, composites, glass-ceramics, and metal alloys (e.g., magnesium alloys, titanium alloys, etc.). Suitable materials are described, for example, in Prakasam et al. (J. Funct. Biomater. (2017) 8 (4): E44), incorporated herein by reference. In certain embodiments, the biocompatible material comprises hydroxyapatite.

In certain embodiments, the biocompatible material of the scaffold (e.g., 3D printed scaffold) is a polymer. The scaffold of the instant invention may comprise any biocompatible polymer. The polymer may be biodegradable or non-biodegradable. The polymer may by hydrophobic, hydrophilic, or amphiphilic. In a particular embodiment, the polymer is hydrophobic. In a particular embodiment, the polymer is hydrophilic. In certain embodiments, the scaffold comprises at least one hydrophilic polymer and at least one hydrophobic polymer. The polymers of the instant invention may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, polyhydroxybutyrate (PHB), polyhydroxyvalerate (PHV), poly(3-hydroxybutyrate-co-3-hydroxyvalerate) (PHBV), PCL/calcium carbonate, and/or poly(styrene). In certain embodiments, the hydrophobic polymer comprises a polymer selected from the group consisting of polylactic acid (PLA), poly glycolic (PGA), poly(lactic-co-glycolic acid) (PLGA), polyhydroxybutyrate (PHB), polyhydroxyvalerate (PHV), copolymers of PHB and PHV, and polycaprolactone. In a particular embodiment, the hydrophobic polymer comprises polycaprolactone.

Examples of hydrophilic polymers include, without limitation: polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO. In a particular embodiment, the hydrophilic polymer comprises hyaluronic acid and/or gelatin. In certain embodiments, the hydrophilic polymers are methacrylated. In a particular embodiment, the hydrophilic polymer comprises methacrylated hyaluronic acid and/or methacrylated gelatin.

Amphiphilic copolymers or polymer composites may comprise a hydrophilic polymer (e.g., segment) and a hydrophobic polymer (e.g., segment) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

In certain embodiments, the scaffolds of the instant invention are mineralized (e.g., comprise minerals and/or coated with minerals). Mineralization, for example, with hydroxyapatite, can enhance the adhesion of osteogenic precursor cells in vitro and in vivo (Duan, et al., Biomacromolecules (2017) 18:2080-2089). In certain embodiments, the scaffolds are coated with and/or comprise Ca, P, and/or O (e.g., immersed in simulated body fluid (SBF) for mineralization (e.g., a solution comprising NaCl, CaCl₂), NaH₂PO₄, and NaHCO₃). In certain embodiments, the scaffolds are coated with and/or comprise hydroxyapatite, fluorapatite, or chlorapatite, particularly hydroxyapatite. In a particular embodiment, the scaffolds of the instant invention further comprise hydroxyapatite. In certain embodiments, the hydroxyapatite is in the form of nanocrystals. In certain embodiments, the printing materials may comprise hydroxyapatite and a biocompatible material (e.g., polymer). In certain embodiments, the hydroxyapatite is contained within the hydrophobic polymer (e.g., polycaprolactone).

In certain embodiments, the scaffolds of the instant invention comprise one or more hydrophobic portions and one or more hydrophilic portions. In certain embodiments, the hydrophobic portion comprises at least one hydrophobic biocompatible material such as a hydrophobic polymer. In certain embodiments, the hydrophilic portion comprises at least one hydrophilic biocompatible material such as a hydrophilic polymer. In certain embodiments, the hydrophobic portion comprises polycaprolactone and hydroxyapatite. In certain embodiments, the hydrophilic portion comprises hyaluronic acid (e.g., methacrylated hyaluronic acid) and gelatin (e.g., methacrylated gelatin). In certain embodiments, the hydrophobic portion (e.g., PCL/hydroxyapatite) is printed in spaced lines and/or a frame, and the hydrophilic portion (e.g., hyaluronic acid/gelatin) is printed in between the lines and/or frame. In certain embodiments, the 3D printed scaffold is printed in a layer-by-layer manner, particularly wherein the scaffold is rotated 90° at each successive layer to create a grid pattern in the scaffold.

In certain embodiments, the hydrogel and/or structure/scaffold of the instant invention comprises at least one therapeutic. In certain embodiments, the hydrogel and/or structure/scaffold of the instant invention comprises at least one antimicrobial and/or cytokine. The contents (e.g., at least one antimicrobial (e.g., antibiotic) and/or cytokine) of the hydrogel may be the same or different than the one(s) contained within the structure/scaffold. The antimicrobial (e.g., antibiotic) and/or cytokine may be covalently attached to the hydrogel (e.g., alginate) and/or structure/scaffold, either directly or through a linker (e.g., as described herein). In certain embodiments, the hydrogel will comprise unconjugated hydrogel and hydrogel covalently attached to the antimicrobial and/or cytokine.

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antimicrobial peptide (e.g., LL-37 or a fragment or derivative thereof (e.g., those provided in Wang et al. (2019) Adv. Exp. Med. Biol., 1117:215-240, incorporated herein by reference). In a particular embodiment, the antimicrobial is a small molecule antibiotic. Examples of antibiotics include, without limitation, beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, ansamycins (e.g., rifamycins, rifampin), aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin). In certain embodiments, the hydrogel and/or structure/scaffold comprises daptomycin and/or rifampin.

In certain embodiments, if the antimicrobial (e.g., antibiotic) is hydrophobic, it is contained within the hydrophobic portion of the hydrogel and/or structure/scaffold. In certain embodiments, if the antimicrobial (e.g., antibiotic) is hydrophilic, it is contained within the hydrophilic portion of the hydrogel and/or structure/scaffold.

As stated hereinabove, the hydrogel and/or structure/scaffold may comprise at least one cytokine. The cytokine may be pro- or anti-inflammatory. In certain embodiments, the hydrogel and/or structure/scaffold comprises pro-inflammatory cytokines. Pro-inflammatory cytokines include, without limitation: IL-1, IL-6, IL-8, IL-12, IFN-γ, IL-18, TNF, macrophage colony-stimulating factor (M-CSF) and IFN-γ. In certain embodiments, the pro-inflammatory cytokines are M-CSF and/or IFN-γ.

In certain embodiments, the hydrogel and/or structure/scaffold may also further comprise at least one analgesic.

The hydrogel and/or structure/scaffold of the instant invention may further comprise and/or encapsulate cells. In certain embodiments, the hydrogel and/or structure/scaffold further comprises macrophage. In certain embodiments, the hydrogel and/or structure/scaffold further comprises neutrophil. In certain embodiments, the cells (e.g., macrophage) are autologous to the subject to be treated. The hydrogel and/or structure/scaffold may comprise and/or encapsulate any cell type. In certain embodiments, the cells are immune cells such as, but not limited to, T cells, B cells, NK cells, macrophages, neutrophils, dendritic cells and modified forms of these cells and various combinations thereof. In certain embodiments, the cells are phagocytic. In certain embodiments, the cells are macrophage (e.g., pro-inflammatory macrophage). The cells (e.g., macrophage) may be autologous or allogenic. The cells (e.g., macrophage) may be activated or non-activated. The cells (e.g., macrophage) may be cultured on the hydrogel and/or structure/scaffold (e.g., the cells may be cultured for sufficient time to allow for growth in and/or on the hydrogel and/or structure/scaffold). For example, the cells may be cultured for 1 day, 2 days, 3 days, 4 days, 5 days, or more. In certain embodiments, the cells (e.g., macrophage) are printed onto the surface of or within the hydrogel and/or structure/scaffold.

In accordance with the instant invention, functionalized therapeutics are provided. In certain embodiments, the therapeutic is functionalized with a compound comprising a reactant of a click chemistry reaction, in particular a reactant which complements the reactant attached to the hydrogel. In certain embodiments, the click chemistry reaction is an azide-alkyne cycloaddition. In certain embodiments, the therapeutic is functionalized with an azide group (N₃). In certain embodiments, the therapeutic is functionalized with an alkyne group (carbon-carbon triple bond). In certain embodiments, the reactant of the click chemistry reaction is covalently attached to the therapeutic directly or via a linker. In certain embodiments, a compound comprising the reactant of the click chemistry reaction is covalently attached to the therapeutic directly or via a linker. Compositions comprising the functionalized therapeutic and at least one pharmaceutically acceptable carrier are also encompassed by the instant invention.

In certain embodiments, the therapeutic is covalently attached directly or via a linker to a compound comprising an alkyne group. In certain embodiments, the therapeutic is covalently attached directly or via a linker to a compound comprising dibenzocyclooctyne (DBCO). In certain embodiments, the therapeutic is covalently attached via a linker to dibenzocyclooctyne (DBCO). In certain embodiments, the functionalized therapeutic comprises an antimicrobial (e.g., an antibiotic (e.g., daptomycin or vancomycin)) covalently attached via a linker to dibenzocyclooctyne (DBCO). In certain embodiments, the functionalized therapeutic is covalently attached directly or via a linker to a compound comprising an alkyne group via an —NH₂ group of the therapeutic. In certain embodiments, the functionalized therapeutic is vancomycin, wherein the compound comprising an alkyne group (e.g., DBCO) is covalently attached directly or via a linker to the —NH₂ group (e.g., of the tetrahydropyran/oxane group) of vancomycin. In certain embodiments, the linker comprises polyethylene glycol.

In certain embodiments, the therapeutic is covalently attached directly or via a linker to a compound comprising an azide group. In certain embodiments, the therapeutic is covalently attached directly or via a linker to an azide group. In certain embodiments, the therapeutic is covalently attached via a linker to an azide group. In certain embodiments, the functionalized therapeutic comprises an antimicrobial (e.g., an antibiotic (e.g., daptomycin or vancomycin)) covalently attached via a linker to an azide group. In certain embodiments, the functionalized therapeutic is covalently attached directly or via a linker to a compound comprising an azide group via an —NH₂ group of the therapeutic. In certain embodiments, the functionalized therapeutic is vancomycin, wherein the compound comprising an azide is covalently attached directly or via a linker to the —NH₂ group (e.g., of the tetrahydropyran/oxane group) of vancomycin. In certain embodiments, the linker comprises polyethylene glycol.

Therapeutics of the instant invention may include, without limitation, drugs, pharmaceuticals, biologics, growth factor, cytokines, chemokines, antibodies, antibody fragments, small molecules, peptides, proteins, nucleic acid molecules, DNA, RNA, and other known biologic substances. In certain embodiments, the therapeutic is an antimicrobial. In certain embodiments, the therapeutic is a cytokine. In certain embodiments, the therapeutic is an analgesic.

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antimicrobial peptide (e.g., LL-37 or a fragment or derivative thereof (e.g., those provided in Wang et al. (2019) Adv. Exp. Med. Biol., 1117:215-240, incorporated herein by reference). In a particular embodiment, the antimicrobial is a small molecule antibiotic. Examples of antibiotics include, without limitation, beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, ansamycins (e.g., rifamycins, rifampin), aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin). In certain embodiments, the therapeutic is an antibiotic. In certain embodiments, the therapeutic is vancomycin. In certain embodiments, the therapeutic is daptomycin.

As stated hereinabove, the therapeutic may be a cytokine. The cytokine may be pro- or anti-inflammatory. In certain embodiments, the therapeutic is a pro-inflammatory cytokine. Pro-inflammatory cytokines include, without limitation: IL-1, IL-6, IL-8, IL-12, IFN-γ, IL-18, TNF, macrophage colony-stimulating factor (M-CSF) and IFN-γ. In certain embodiments, the pro-inflammatory cytokines are M-CSF and/or IFN-γ.

In accordance with the instant invention, functionalized vesicles are provided. In certain embodiments, the functionalized vesicles are lipid vesicles. In certain embodiments, the vesicles are nanoparticles or lipid nanoparticles. In certain embodiments, the functionalized vesicles are liposomes and/or micelles (e.g., polymeric micelle). In certain embodiments, the vesicle is functionalized with a compound comprising a reactant of a click chemistry reaction, in particular a reactant which complements the reactant attached to the hydrogel. In certain embodiments, the click chemistry reaction is an azide-alkyne cycloaddition. In certain embodiments, the vesicle is functionalized with an azide group (N₃). In certain embodiments, the vesicle is functionalized with an alkyne group (carbon-carbon triple bond). In certain embodiments, the reactant of the click chemistry reaction is covalently attached to the vesicle directly or via a linker. In certain embodiments, a compound comprising the reactant of the click chemistry reaction is covalently attached to the vesicle directly or via a linker. In certain embodiments, the vesicle is functionalized on its surface (exterior). Compositions comprising the functionalized vesicle and at least one pharmaceutically acceptable carrier are also encompassed by the instant invention.

In certain embodiments, the vesicles comprise at least one lipid, phospholipid, and/or amphiphilic polymer. In certain embodiments, the vesicles comprise cholesterol. In certain embodiments, the vesicles comprise one or more ionizable lipids (e.g., DLin-MC3-DMA, ALC-0315, and/or SM-102). In certain embodiments, the vesicles comprise one or more pegylated lipids (e.g., PEG2000-C-DMG, ALC-0159, and/or PEG2000-DMG). In certain embodiments, the vesicles comprise one or more phospholipid. Examples of phospholipids include, without limitation: phosphatidylcholines, phosphatidylethanolamines, phosphatidylserines, and phosphatidylglycerols. Other examples of phospholipids include, without limitation: distearoyl phosphatidylcholine (DSPC), distearoyl phosphatidylethanolamine (DSPE), distearoyl phosphatidylglycerol (DSPG), dioleoyl phosphatidylethanolamine (DOPE), dioleoyl phosphatidylcholine (DOPC), dioleoyl phosphatidylserine (DOPS), dimyristoyl phosphatidylglycerol (DMPG), dimyristoyl phosphatidylethanolamine (DMPE), dimyristoyl phosphatidylcholine (DMPC), distearoyl phosphatidylglycerol (DSPG), 1,2-dilauroyl-sn-glycero-3-phosphoethanolamine (DLPE), 1,2-dilauroyl-sn-glycero-3-phosphocholine (DLPC), 1,2-dilauroyl-sn-glycero-3-phosphatidylglycerol (DLPG), and derivatives thereof (e.g., PEG derivatives such as DSPE-PEG). In certain embodiments, the vesicle comprises ALC-0315, DSPC, cholesterol, and ALC-0159. In certain embodiments, the vesicle comprises SM-102, DSPC, cholesterol. PEG2000-DMG. In certain embodiments, the vesicle comprises distearoyl phosphatidylcholine (DSPC). In certain embodiments, the vesicle comprises distearoyl phosphatidylethanolamine (DSPE).

In certain embodiments, the vesicle is covalently attached directly or via a linker to a compound comprising an alkyne group. In certain embodiments, the vesicle is covalently attached directly or via a linker to a compound comprising dibenzocyclooctyne (DBCO). In certain embodiments, the vesicle is covalently attached via a linker to dibenzocyclooctyne (DBCO). In certain embodiments, the functionalized vesicle is covalently attached directly or via a linker to a compound comprising an alkyne group via an amine group of the vesicle (e.g., a component (e.g., phospholipid) of the vesicle). In certain embodiments, the functionalized vesicle is covalently attached directly or via a linker to a compound comprising an alkyne group via an amine group linked to the phosphate group of the phospholipid. In certain embodiments, the linker comprises polyethylene glycol.

In certain embodiments, the vesicle is covalently attached directly or via a linker to a compound comprising an azide group. In certain embodiments, the vesicle is covalently attached directly or via a linker to an azide group. In certain embodiments, the vesicle is covalently attached via a linker to an azide group. In certain embodiments, the functionalized vesicle is covalently attached directly or via a linker to a compound comprising an azide group via an amine group of the vesicle (e.g., a component (e.g., phospholipid) of the vesicle). In certain embodiments, the functionalized vesicle is covalently attached directly or via a linker to a compound comprising an azide group via an amine group linked to the phosphate group of the phospholipid. In certain embodiments, the linker comprises polyethylene glycol.

The functionalized vesicle of the instant invention may comprise and/or encapsulate at least one therapeutic. Therapeutics of the instant invention may include, without limitation, drugs, pharmaceuticals, small molecules, peptides, proteins, DNA, RNA, and other known biologic substances. In certain embodiments, the therapeutic is an antimicrobial. In certain embodiments, the therapeutic is a cytokine. In certain embodiments, the therapeutic is an analgesic.

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antimicrobial peptide (e.g., LL-37 or a fragment or derivative thereof (e.g., those provided in Wang et al. (2019) Adv. Exp. Med. Biol., 1117:215-240, incorporated herein by reference). In a particular embodiment, the antimicrobial is a small molecule antibiotic. Examples of antibiotics include, without limitation, beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, ansamycins (e.g., rifamycins, rifampin), aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin). In certain embodiments, the therapeutic is an antibiotic. In certain embodiments, the therapeutic is vancomycin. In certain embodiments, the therapeutic is daptomycin.

As stated hereinabove, the therapeutic may be a cytokine. The cytokine may be pro- or anti-inflammatory. In certain embodiments, the vesicle comprises pro-inflammatory cytokines. Pro-inflammatory cytokines include, without limitation: IL-1, IL-6, IL-8, IL-12, IFN-γ, IL-18, TNF, macrophage colony-stimulating factor (M-CSF) and IFN-γ. In certain embodiments, the pro-inflammatory cytokines are M-CSF and/or IFN-γ.

In accordance with the instant invention, methods of delivering a therapeutic to a subject in need thereof are provided. In certain embodiments, the method comprises a) administering or implanting a functionalized hydrogel of the instant invention to the subject, and b) administering a functionalized therapeutic or functionalized vesicle of the instant invention to the subject. In certain embodiments, the functionalized hydrogel coats and/or is contained within a scaffold. In certain embodiments, step b) is performed at least once after step a), as needed. In certain embodiments, step b) is performed more than once at different timepoints after step a). For example, step b) may be performed once a week, once every two weeks, once every three weeks, once every four weeks, once every month, once every two months, or once every three months or longer as needed for treatment of the subject. In certain embodiments, step b) comprises systemic administration. In certain embodiments, step b) comprises intravenous and/or intraperitoneal administration. In certain embodiments, step b) comprises direct administration or injection to or near the implant of step a) (e.g., locally).

In certain embodiments, the functionalized hydrogel comprises alginate. In certain embodiments, the hydrogel is functionalized with an azide group. In certain embodiments, the hydrogel is functionalized with an alkyne group. In certain embodiments, the hydrogel is functionalized with DBCO.

In certain embodiments, the functionalized therapeutic is an antimicrobial (e.g., antibiotic). In certain embodiments, the therapeutic is functionalized with an azide group. In certain embodiments, the therapeutic is functionalized with an alkyne group. In certain embodiments, the therapeutic is functionalized with DBCO.

In certain embodiments, the functionalized vesicle comprises and/or encapsulates an antimicrobial (e.g., antibiotic). In certain embodiments, the vesicle is functionalized with an azide group. In certain embodiments, the vesicle is functionalized with an alkyne group. In certain embodiments, the vesicle is functionalized with DBCO.

In certain embodiments, the functionalized hydrogel comprises alginate covalently attached to DBCO via a linker and the functionalized therapeutic is an antimicrobial (e.g., antibiotic) covalently attached directly or via a linker to an azide group. In certain embodiments, the therapeutic is vancomycin covalently attached via a linker an azide.

In certain embodiments, the functionalized hydrogel comprises alginate covalently attached to an azide via a linker and the functionalized therapeutic is an antimicrobial (e.g., antibiotic) covalently attached directly or via a linker to an alkyne group. In certain embodiments, the antimicrobial (e.g., antibiotic) is covalently attached via a linker to DBCO. In certain embodiments, the therapeutic is vancomycin covalently attached via a linker to DBCO.

In certain embodiments, the functionalized hydrogel comprises alginate covalently attached to DBCO via a linker and the functionalized vesicle comprises and/or encapsulates an antimicrobial (e.g., antibiotic) and the vesicle is covalently attached directly or via a linker to an azide group. In certain embodiments, the vesicle is a liposome (e.g., a DSPE liposome). In certain embodiments, the vesicle is a nanoparticle. In certain embodiments, the vesicle comprises and/or encapsulates daptomycin.

In certain embodiments, the functionalized hydrogel comprises alginate covalently attached to an azide via a linker and the functionalized vesicle comprises and/or encapsulates an antimicrobial (e.g., antibiotic) and the vesicle is covalently attached directly or via a linker to an alkyne group (e.g., DBCO). In certain embodiments, the vesicle is a liposome (e.g., a DSPE liposome). In certain embodiments, the vesicle is a nanoparticle. In certain embodiments, the vesicle comprises and/or encapsulates daptomycin.

In certain embodiments, the method comprises implanting or inserting a hydrogel and/or scaffold (e.g., 3D printed scaffold) of the instant invention into a hole or cavity in the subject (e.g., in a bone of the subject such as in the skull of the subject). In certain embodiments, the hydrogel and/or scaffold is inserted on top of an implant or prosthetic (e.g., on top of a bone flap). In certain embodiments, the hydrogel and/or scaffold is implanted or inserted into a hole or cavity in the skull caused by the removal of bone during a craniotomy or craniectomy. The methods of the instant invention may further comprise the removal of a portion of the skull for the insertion or implantation of the hydrogel and/or scaffold. The methods of the instant invention may optionally comprise replacing the cranial bone flap. In certain embodiments, the subject to be treated by the instant methods have undergone a craniotomy or craniectomy (e.g., a depressurization craniotomy or craniectomy) to treat a brain tumor, to treat epilepsy, or to treat a head wound or traumatic brain injury (e.g., military personnel).

The methods of the instant invention may further comprise scanning the hole or cavity in the brain (e.g., by a CT scan) to determine the size and shape of the scaffold. The methods may further comprise synthesizing the scaffold (e.g., with a high-resolution 3D printer (e.g., 3D-Bioplotter®)).

In accordance with another aspect of the instant invention, methods of treating, inhibiting, and/or preventing a microbial or bacterial infection (e.g., biofilm infection) are provided. In certain embodiment, the method treats, inhibits, and/or prevents an infection associated with a prosthetic or surgical implant. In certain embodiments, the method treats, inhibits, and/or prevents an infection associated with a craniotomy or craniectomy. In certain embodiments, the method treats, inhibits, and/or prevents a cranial bone flap infection. In certain embodiments, the method treats, inhibits, and/or prevents the recurrence of infection after the surgical removal of an infected prosthetic or implant (e.g., cranial bone flap).

In certain embodiments, the method comprises a) administering or implanting a functionalized hydrogel of the instant invention to the subject, and b) administering a functionalized therapeutic or functionalized vesicle of the instant invention to the subject. In certain embodiments, the functionalized hydrogel coats and/or is contained within a scaffold. In certain embodiments, step b) is performed at least once after step a), as needed. In certain embodiments, step b) is performed more than once at different timepoints after step a). For example, step b) may be performed once a week, once every two weeks, once every three weeks, once every four weeks, once every month, once every two months, or once every three months or longer as needed for treatment of the subject. In certain embodiments, step b) comprises systemic administration. In certain embodiments, step b) comprises intravenous and/or intraperitoneal administration. In certain embodiments, step b) comprises direct administration or injection to or near the implant of step a) (e.g., locally).

In certain embodiments, the functionalized hydrogel comprises alginate. In certain embodiments, the hydrogel is functionalized with an azide group. In certain embodiments, the hydrogel is functionalized with an alkyne group. In certain embodiments, the hydrogel is functionalized with DBCO.

In certain embodiments, the functionalized therapeutic is an antimicrobial (e.g., antibiotic). In certain embodiments, the therapeutic is functionalized with an azide group. In certain embodiments, the therapeutic is functionalized with an alkyne group. In certain embodiments, the therapeutic is functionalized with DBCO.

In certain embodiments, the functionalized vesicle comprises and/or encapsulates an antimicrobial (e.g., antibiotic). In certain embodiments, the vesicle is functionalized with an azide group. In certain embodiments, the vesicle is functionalized with an alkyne group. In certain embodiments, the vesicle is functionalized with DBCO.

In certain embodiments, the functionalized hydrogel comprises alginate covalently attached to DBCO via a linker and the functionalized therapeutic is an antimicrobial (e.g., antibiotic) covalently attached directly or via a linker to an azide group. In certain embodiments, the therapeutic is vancomycin covalently attached via a linker an azide.

In certain embodiments, the functionalized hydrogel comprises alginate covalently attached to an azide via a linker and the functionalized therapeutic is an antimicrobial (e.g., antibiotic) covalently attached directly or via a linker to an alkyne group. In certain embodiments, the antimicrobial (e.g., antibiotic) is covalently attached via a linker to DBCO. In certain embodiments, the therapeutic is vancomycin covalently attached via a linker to DBCO.

In certain embodiments, the functionalized hydrogel comprises alginate covalently attached to DBCO via a linker and the functionalized vesicle comprises and/or encapsulates an antimicrobial (e.g., antibiotic) and the vesicle is covalently attached directly or via a linker to an azide group. In certain embodiments, the vesicle is a liposome (e.g., a DSPE liposome). In certain embodiments, the vesicle is a nanoparticle. In certain embodiments, the vesicle comprises and/or encapsulates daptomycin.

In certain embodiments, the functionalized hydrogel comprises alginate covalently attached to an azide via a linker and the functionalized vesicle comprises and/or encapsulates an antimicrobial (e.g., antibiotic) and the vesicle is covalently attached directly or via a linker to an alkyne group (e.g., DBCO). In certain embodiments, the vesicle is a liposome (e.g., a DSPE liposome). In certain embodiments, the vesicle is a nanoparticle. In certain embodiments, the vesicle comprises and/or encapsulates daptomycin.

In certain embodiments, the method comprises implanting or inserting a hydrogel and/or scaffold (e.g., 3D printed scaffold) of the instant invention into a hole or cavity in the subject (e.g., in a bone of the subject such as in the skull of the subject). In certain embodiments, the hydrogel and/or scaffold is inserted on top of an implant or prosthetic (e.g., on top of a bone flap). In certain embodiments, the scaffold is implanted or inserted into a hole or cavity in the skull caused by the removal of bone during a craniotomy or craniectomy. The methods of the instant invention may further comprise the removal of a portion of the skull for the insertion or implantation of the hydrogel and/or scaffold. The methods of the instant invention may optionally comprise replacing the cranial bone flap. In certain embodiments, the subject to be treated by the instant methods have undergone a craniotomy or craniectomy (e.g., a depressurization craniotomy or craniectomy) to treat a brain tumor, to treat epilepsy, or to treat a head wound or traumatic brain injury (e.g., military personnel).

The methods of the instant invention may further comprise scanning the hole or cavity in the brain (e.g., by a CT scan) to determine the size and shape of the scaffold. The methods may further comprise synthesizing the scaffold (e.g., with a high-resolution 3D printer (e.g., 3D-Bioplotter®)).

The components (e.g., functionalized hydrogel, functionalized therapeutic, and/or functionalized vesicle) of the instant invention (optionally in a composition) can be administered to an animal, in particular a mammal, more particularly a human, (e.g., in order to treat/inhibit/prevent the disease or disorder (e.g., infection)). The pharmaceutical compositions of the instant invention may also comprise at least one other compound or therapeutic agent such as an antiviral agent or antimicrobial. The additional compound may also be administered in a separate pharmaceutical composition from the compositions of the instant invention. The pharmaceutical compositions may be administered at the same time or at different times (e.g., sequentially).

The dosage ranges for the administration of the compositions of the invention (e.g., functionalized therapeutic and/or functionalized vesicle) are those large enough to produce the desired effect (e.g., curing, relieving, treating, and/or preventing the disease or disorder, the symptoms of it, or the predisposition towards it). The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The compositions described herein will generally be administered to a patient as a pharmaceutical composition. The term “patient” as used herein refers to human or animal subjects. These compositions may be employed therapeutically, under the guidance of a physician.

The pharmaceutical compositions of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the components in the chosen medium may be varied and the medium may be chosen based on the desired route of administration of the pharmaceutical composition. Except insofar as any conventional media or agent is incompatible with the component to be administered, its use in the pharmaceutical composition is contemplated.

The dose and dosage regimen of compositions according to the invention that are suitable for administration to a particular patient may be determined by a physician considering the patient's age, sex, weight, general medical condition, and the specific condition for which the components are being administered and the severity thereof. The physician may also take into account the route of administration, the pharmaceutical carrier, and the component's biological activity.

Selection of a suitable pharmaceutical composition will also depend upon the mode of administration chosen. For example, the components of the invention (e.g., functionalized therapeutic and/or functionalized vesicle) may be administered by direct injection or to the bloodstream (e.g., intravenously). In this instance, a pharmaceutical composition comprises the component dispersed in a medium that is compatible with the site of injection.

Components of the instant invention may be administered by any method. For example, the components of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the component (e.g., functionalized therapeutic and/or functionalized vesicle) is administered parenterally. In a particular embodiment, the component (e.g., functionalized therapeutic and/or functionalized vesicle) is administered intravenously. In a particular embodiment, the component (e.g., functionalized therapeutic and/or functionalized vesicle) is administered by direct injection. In a particular embodiment, the component is administered orally, intramuscularly, subcutaneously, or to the bloodstream (e.g., intravenously). In a particular embodiment, the component (e.g., functionalized therapeutic and/or functionalized vesicle) is administered intramuscularly or subcutaneously. Pharmaceutical compositions for injection are known in the art. If injection is selected as a method for administering the component, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a component of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. In a particular embodiment, the components of the instant invention, due to their long-acting therapeutic effect, need only be administered once to a subject.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of a component (e.g., functionalized therapeutic and/or functionalized vesicle) may be determined by evaluating their toxicity in animal models. Various concentrations of components in pharmaceutical composition may be administered to mice, and the minimal and maximal dosages may be determined based on the beneficial results and side effects observed as a result of the treatment. Appropriate dosage unit may also be determined by assessing the efficacy of the treatment in combination with other standard drugs. The dosage units may be determined individually or in combination with each treatment according to the effect detected.

The pharmaceutical composition comprising the components (e.g., functionalized therapeutic and/or functionalized vesicle) may be administered at appropriate intervals until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCI, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N. Y.; and Kibbe, et al., Eds., Handbook of Pharmaceutical Excipients, American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, treat, or lessen the symptoms of a particular disorder or disease. For example, the treatment of an infection herein may refer to reducing, curing, and/or relieving the infection, the symptom(s) of it, or the predisposition towards it.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 4,000, or less than 2,000 Da). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following examples illustrate certain embodiments of the invention. They are not intended to limit the invention in any way.

### Example 1

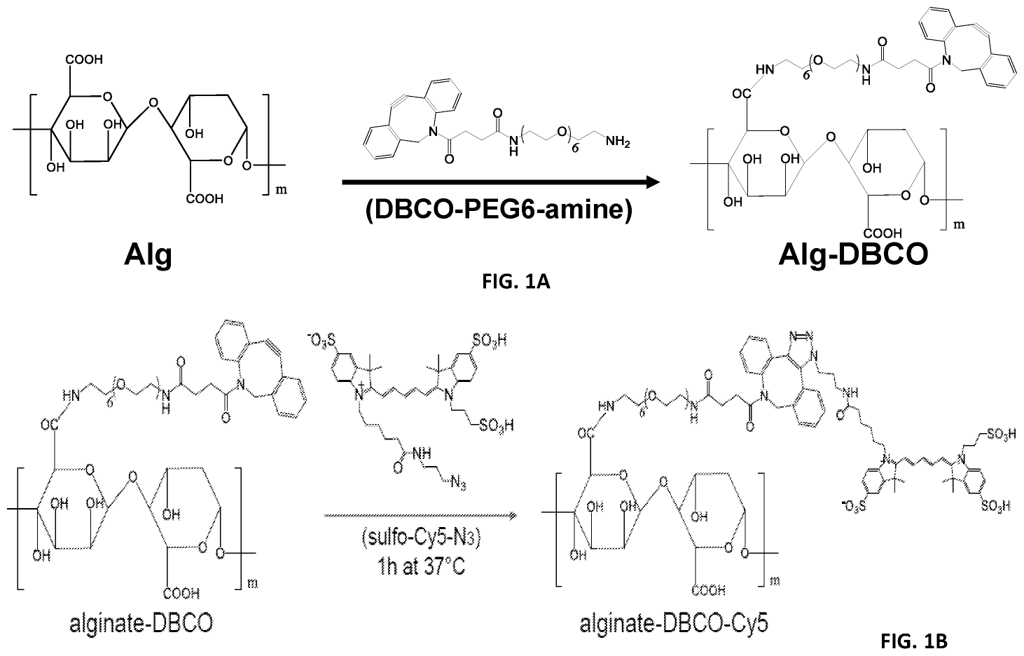

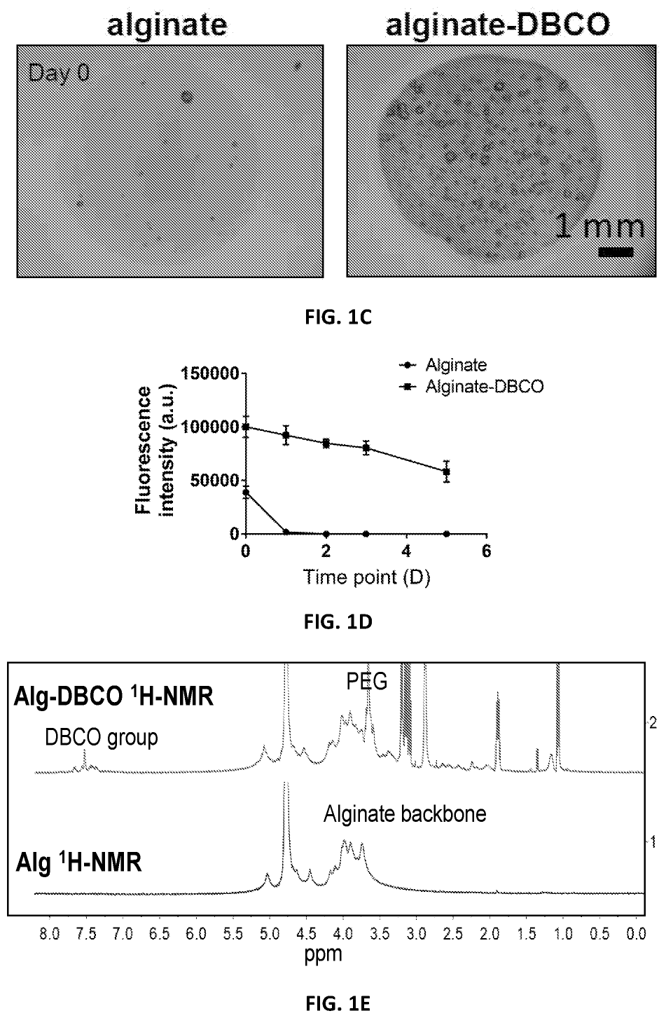

Alginate was modified with dibenzocyclooctyne (DBCO). Specifically, alginate was reacted with DBCO-PEG₆-amine to produce alginate-DBCO. A schematic of an example synthesis scheme is provided in FIG. 1A. Briefly, 65 mg sodium alginate dissolved in 20 mL H₂O was mixed with 36 mg of DBCO-PEG₆-amine and stirred at room temperature for 20 minutes. Then 1-ethyl-3-(3-dimethylaminopropyl) carbodiimide (EDC) 0.3 mmole (57.6 mg) and N-hydroxysuccinimide (NHS) 0.3 mmole (34.5 mg) was added to the mixture. Final pH was adjusted to 5.5 using HCl solution. The total mixture was stirred at room temperature for 48 hours. After completion of the reaction, the mixture was transferred into 6-8 kDa molecular weight cutoff (MWCO) dialysis bag and dialyzed against water for 3 days. Nuclear magnetic resonance (NMR) confirmed the synthesis of alginate-DBCO (FIG. 1E).

Alginate-DBCO is reactive with N₃ groups. This functionality was demonstrated by reacting alginate-DBCO with sulfo-Cy5-N₃ to yield alginate-DBCO-Cy5. A schematic of an example synthesis scheme is provided in FIG. 1B. Briefly, alginate and alginate-DBCO were prepared as 3% (w/v) solution and then mixed with 0.5% calcium chloride solution at 7:3 volume ratio to prepare injectable hydrogel. Then both injectable hydrogels were injected into a 6 mm diameter mold (30 μL volume). Molded hydrogels were immersed in 2.5% calcium chloride solution for 10 minutes. Then the two types of hydrogel scaffolds were transferred into each well of a 24 well plate and incubated with 10 μg/ml of sulfo-Cy5-N₃ solution (1 mL for each scaffold) for 1 hour followed by washing with PBS. Then the fluorescence intensity inside the scaffolds were determined by a plate reader. The scaffolds were then incubated in H₂O for 5 days with fluorescence intensity (Ex/Em) monitored at pre-determined time points. As seen in FIG. 1C, alginate-DBCO successfully reacted with sulfo-Cy5-N₃, but unmodified alginate did not. FIG. 1D provides a graph of the measured fluorescence.

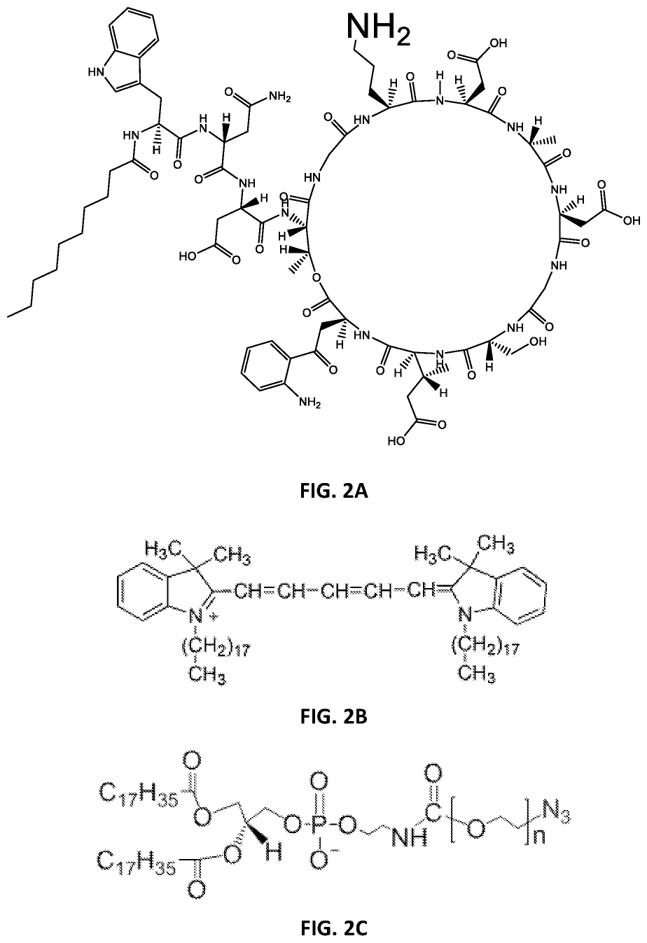

N₃-functionalzied liposomes were also synthesized. More specifically, liposomes of distearoylphosphatidylethanolamine (DSPE) functionalized with N₃ (FIG. 2C) were synthesized which encapsulated daptomycin (FIG. 2A) and contained 1,1′-dioctadecyl-3,3,3′,3′-tetramethylindodicarbocyanine (DiD) (FIG. 2B) lipophilic dye with a ratio (w/w) of DSPE-PEG-N₃:daptomycin:DiD of 10:1:0.1. Briefly, the liposomes were synthesized by a thin film hydration process. Lipid, drug, and dye were added to a solution of methanol and chloroform. The solution was evaporated, causing film formation. The film was then hydrated and sonicated to yield the functionalized liposomes. Dug encapsulation efficiency was determined to be 70.2%. The daptomycin final concentration was theoretically 0.2 mg/ml with 2 mg/ml lipid.

Notably, the functionalized liposomes may be micelles or be a mixture of liposomes and micelles. For simplicity, the functionalized lipid vesicles (e.g., liposomes and/or micelles; lipid bilayer and/or lipid monolayer) are referred to herein as liposomes.

The N₃ containing liposomes were then characterized. Dynamic light scattering (DLS) was used to determine particle size. As seen in FIG. 3A, the median size of the particles was about 35 nm (diameter). The in vitro binding to alginate scaffolds by the N₃ functionalized liposomes was also determined. Briefly, each scaffold was incubated in 0.5 ml of respective liposome solution (2 mg/ml lipid, Dap 0.2 mg/ml) in 24 well plate at 37° C. for 12 hours. The solution was then removed, and the scaffold was washed in saline for 20 minutes prior to analysis. As seen in FIG. 3B, N₃ functionalized liposomes were only capable of binding alginate-DBCO. FIG. 3C provides a graph of the measured fluorescence (****p<0.0001). FIG. 3D provides the drug release profile for the daptomycin loaded, N₃ functionalized liposomes.

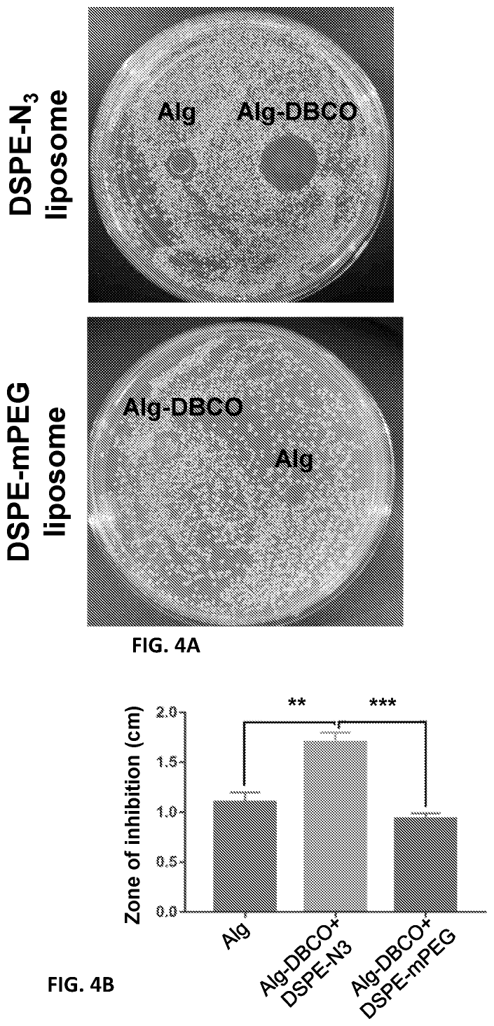

Unmodified alginate scaffolds and alginate-DBCO scaffolds were further characterized after treatment with N₃ functionalized liposomes. Briefly, the normal alginate scaffold and alginate-DBCO scaffold after click reaction with N₃ functionalized liposomes were put on the plate spread with 100 μL of methicillin-resistant Staphylococcus aureus (MRSA) at 10⁵ colony forming units (CFU)/ml. After overnight incubation, the bacterial colony formation was observed (FIG. 4A) and the zone of inhibition was measured (Table 1; FIG. 4B (**p<0.01; ***p<0.001)).

TABLE 1
Zone of inhibition for indicated scaffolds
and liposome combinations.

| Scaffold and Liposome | Zone of Inhibition (cm) |
|---|---|
| Alginate + DSPE-N₃ | 1.1 ± 0.1 |
| Alginate-DBCO + DSPE-N₃ | 1.7 ± 0.15 |
| Alginate + DSPE-mPEG | 0.9 |
| Alginate-DBCO + DSPE-mPEG | NA |

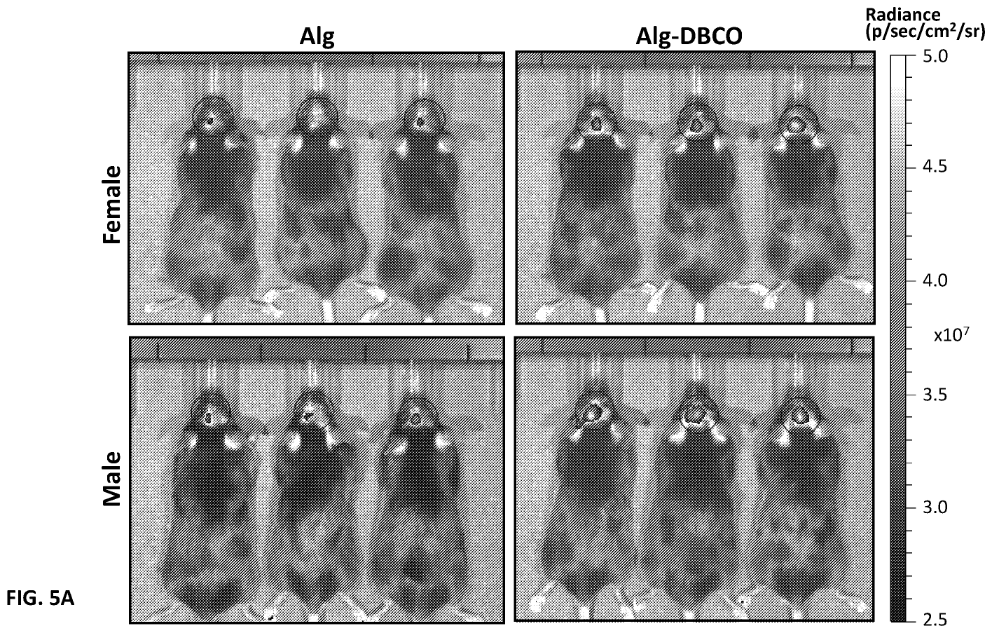

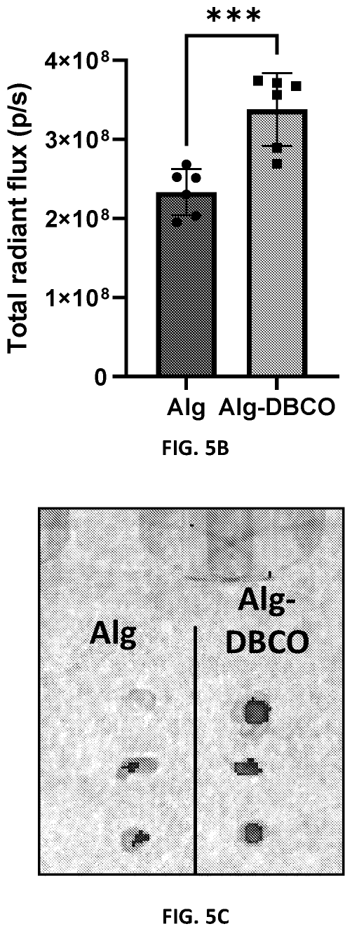

The ability to load the functionalized alginate scaffolds in vivo was also tested. As seen in FIG. 5, specific loading of DBCO-modified alginate hydrogels was achieved in vivo. Briefly, hydrogels-alginate or alginate-DBCO hydrogels-were placed in mice above the bone flap following sterile craniotomy seven days prior to dosing with liposomes. In vivo imaging was performed using an IVIS® Spectrum system (Perkin Elmer; Waltham, MA). Images were collected 24 hours after intravenous (i.v.) dosing of DSPE-N₃ liposomes loaded with DiD dye and daptomycin (IVIS® Ex: 640 nm/Em: 680 nm, 1 sec exposure). FIG. 5A provides images of the in vivo fluorescence. FIG. 5B provides a graph of the quantification of total radiance within marked region of interest (ROI) from images (***p<0.001). FIG. 5C provides images of extracted hydrogels from female mice at day 3 post-dosing.

### Example 2

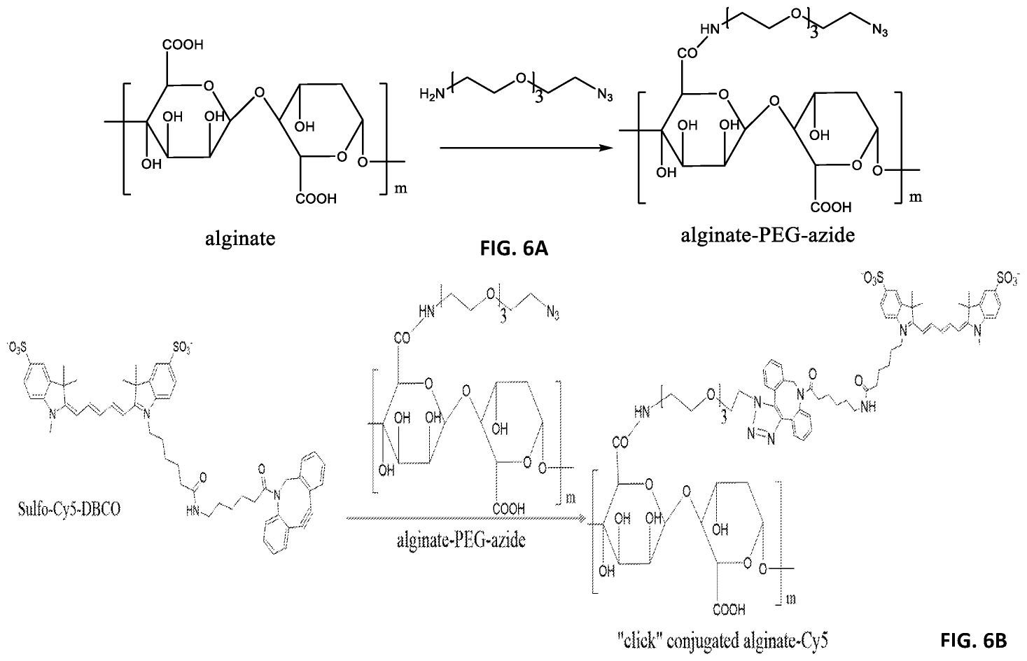

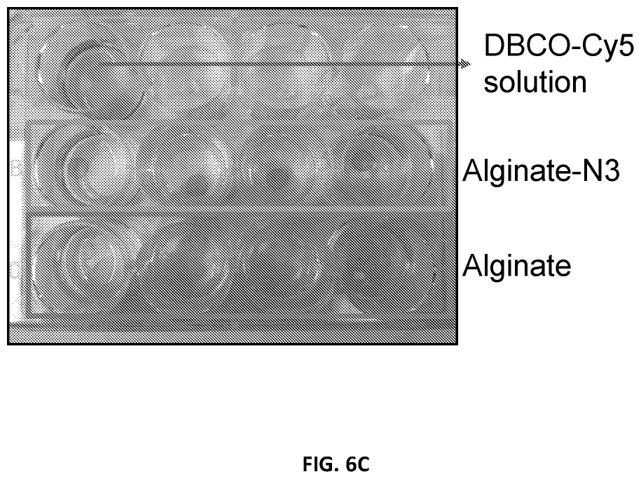

Azide modified alginate hydrogel was also synthesized. An example of a synthesis method for producing azide modified alginate is provided in FIG. 6A. An example of a click reaction between DBCO modified Cy5 and azide modified alginate is provided in FIG. 6B. Alginate and alginate-N₃ hydrogels (n=4) were incubated with 10 μg/ml of sulfo-Cy5-DBCO solution for 12 hours followed by washing with PBS. As seen in FIG. 6C, alginate-N₃—but not unmodified alginate—was successfully modified with Cy5.

DBCO-functionalized liposomes can also be synthesized by methods similar to N₃-functionalized liposomes. More specifically, liposomes of DSPE-PEG-NH₂ can be synthesized which encapsulate daptomycin and contain DiD dye (e.g., with a ratio (w/w) of DSPE-PEG-DBCO:daptomycin:DiD of 10:1:0.1). Briefly, the lipoomes can be synthesized by a thin film hydration process. Lipid, drug, and dye can be added to a solution of methanol and chloroform. The solution can be evaporated, thereby causing film formation. The film can then hydrated and sonicated to yield daptomycin loaded liposomes functionalized with NH₂ groups. DBCO can be added to the liposomes using DBCO-PEG-NHS.

### Example 3

Alginate was modified with DBCO as described above. Specifically, alginate was reacted with DBCO-PEG₆-amine to produce alginate-DBCO. A schematic of an example synthesis scheme is provided in FIG. 1A.

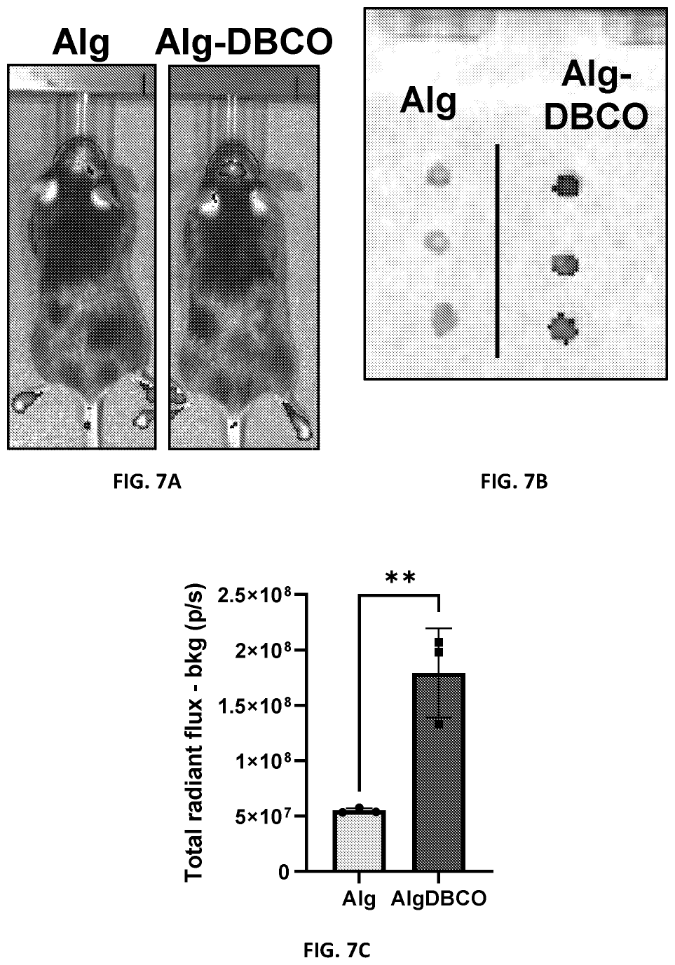

As seen in FIG. 7, specific loading of DBCO-modified alginate hydrogels was achieved in vivo. Briefly, hydrogels-alginate or alginate-DBCO hydrogels-were placed in mice above the bone flap following sterile craniotomy seven days prior to dosing with Cy5-N₃. Hydrogels with incorporated 3D bioprinted hydroxyapatite scaffold were placed above the bone flap following sterile craniotomy 7 days prior to dosing. In vivo imaging was performed using an IVIS® Spectrum system (Perkin Elmer; Waltham, MA). Images were collected 24 hours after intravenous (i.v.) dosing of Cy5-N₃ (Ex: 640 nm/Em: 680 nm, 3 sec exposure). FIG. 7A provides images of the in vivo fluorescence. FIG. 7B provides images of extracted hydrogels from female mice at day 5 post-dosing. FIG. 7C provides a graph of the quantification of total radiance from extracted hydrogels (**p<0.01).

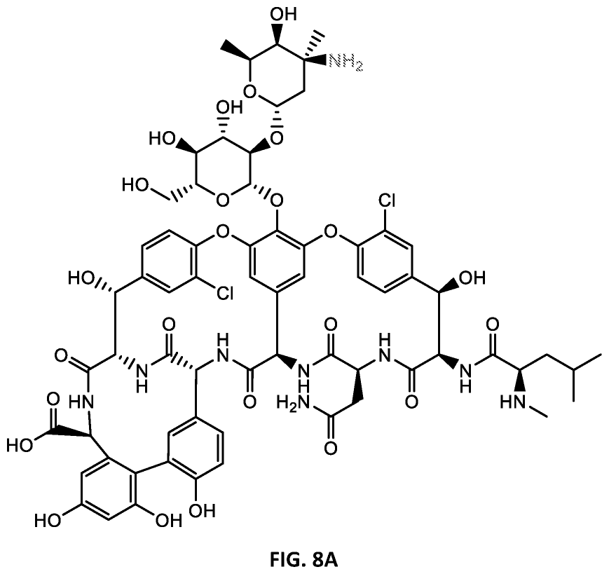

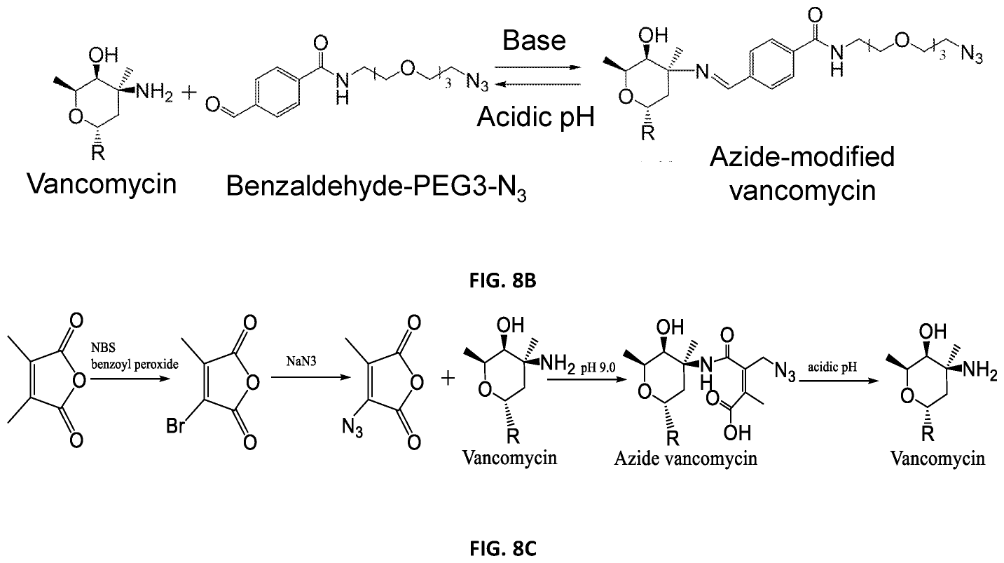

FIG. 8 provides schematics for synthesizing azide modified vancomycin as a prodrug for targeted delivery to the click modified scaffold. FIG. 8A provides the structure of antibiotic vancomycin with the primary amine group for functionalization. FIG. 8B provides an imine based modification approach and FIG. 8C provides an anhydride based modification approach for vancomycin modification.

### Example 4

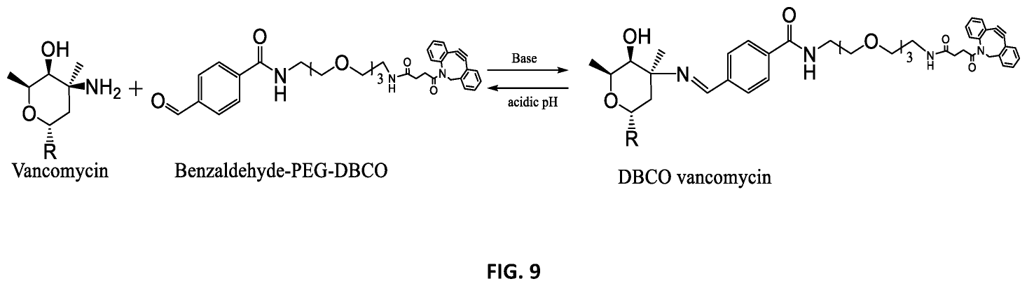

DBCO modified vancomycin can be used as a prodrug for targeted delivery to an azide modified scaffold. The Alg-N₃ can be synthesized as described hereinabove. FIG. 9 provides a schematic for the synthesis of DBCO vancomycin prodrug.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of delivering a therapeutic to a subject in need thereof, said method comprising:
a) administering or implanting a functionalized hydrogel to the subject, wherein said functionalized hydrogel comprises a hydrogel covalently attached directly or via a linker to an azide or alkyne group, and
b) administering a functionalized therapeutic and/or functionalized vesicle to the subject, wherein said functionalized therapeutic comprises a therapeutic covalently attached directly or via a linker to an azide or alkyne group, wherein said functionalized vehicle comprises a vehicle covalently attached directly or via a linker to an azide or alkyne group, and wherein said functionalized vehicle comprises and/or encapsulates a therapeutic,
wherein if said functionalized hydrogel comprises an azide, then said functionalized therapeutic and/or functionalized vesicle comprises an alkyne group, and
wherein if said functionalized hydrogel comprises an alkyne group, then said functionalized therapeutic and/or functionalized vesicle comprises an azide.

2. The method of claim 1, wherein the functionalized hydrogel coats and/or is contained within a scaffold.

3. The method of claim 1, wherein step b) comprises systemic administration.

4. The method of claim 1, wherein step b) comprises intravenous and/or intraperitoneal administration.

5. The method of claim 1, wherein step b) is performed more than once.

6. The method of claim 1, wherein the functionalized hydrogel comprises alginate.

7. The method of claim 1, wherein the hydrogel is functionalized with an azide group.

8. The method of claim 1, wherein the hydrogel is functionalized with an alkyne group.

9. The method of claim 8, wherein the hydrogel is functionalized with DBCO.

10. The method of claim 1, wherein the therapeutic is an antimicrobial or antibiotic.

11. The method of claim 1, wherein the functionalized hydrogel comprises alginate covalently attached to dibenzocyclooctyne (DBCO) via a linker,
wherein the functionalized therapeutic is an antibiotic covalently attached directly or via a linker to an azide group, and
wherein the functionalized vesicle is a liposome covalently attached directly or via a linker to an azide group, and said liposome encapsulates an antibiotic.

12. The method of claim 1, wherein the functionalized hydrogel comprises alginate covalently attached to an azide via a linker,
wherein the functionalized therapeutic is an antibiotic covalently attached directly or via a linker to dibenzocyclooctyne (DBCO), and
wherein the functionalized vesicle is a liposome covalently attached directly or via a linker to dibenzocyclooctyne (DBCO), and said liposome encapsulates an antibiotic.

13. The method of claim 1, wherein said therapeutic is vancomycin or daptomycin.

14. The method of claim 1, wherein said hydrogel is administered or implanted at a site of a prosthetic or surgical implant.

15. The method of claim 1, wherein said hydrogel is administered or implanted at a bone flap.

16. A method of treating, inhibiting, and/or preventing a microbial or bacterial infection in a subject in need thereof, said method comprising:
a) administering or implanting a functionalized hydrogel to the subject, wherein said functionalized hydrogel comprises a hydrogel covalently attached directly or via a linker to an azide or alkyne group, and
b) administering a functionalized therapeutic and/or functionalized vesicle to the subject, wherein said functionalized therapeutic comprises a therapeutic covalently attached directly or via a linker to an azide or alkyne group, wherein said functionalized vehicle comprises a vehicle covalently attached directly or via a linker to an azide or alkyne group, wherein said functionalized vehicle comprises and/or encapsulates a therapeutic, and wherein said therapeutic is an antimicrobial or antibiotic,
wherein if said functionalized hydrogel comprises an azide, then said functionalized therapeutic and/or functionalized vesicle comprises an alkyne group, and
wherein if said functionalized hydrogel comprises an alkyne group, then said functionalized therapeutic and/or functionalized vesicle comprises an azide.

17. The method of claim 16, wherein the functionalized hydrogel coats and/or is contained within a scaffold.

18. The method of claim 16, wherein step b) comprises systemic administration.

19. The method of claim 16, wherein step b) comprises intravenous and/or intraperitoneal administration.

20. The method of claim 16, wherein step b) is performed more than once.

21. The method of claim 16, wherein the functionalized hydrogel comprises alginate.

22. The method of claim 16, wherein the hydrogel is functionalized with an azide group.

23. The method of claim 1, wherein the hydrogel is functionalized with an alkyne group.

24. The method of claim 23, wherein the hydrogel is functionalized with DBCO.

25. The method of claim 16, wherein the functionalized hydrogel comprises alginate covalently attached to dibenzocyclooctyne (DBCO) via a linker,
wherein the functionalized therapeutic is an antibiotic covalently attached directly or via a linker to an azide group, and
wherein the functionalized vesicle is a liposome covalently attached directly or via a linker to an azide group, and said liposome encapsulates an antibiotic.

26. The method of claim 16, wherein the functionalized hydrogel comprises alginate covalently attached to an azide via a linker,
wherein the functionalized therapeutic is an antibiotic covalently attached directly or via a linker to dibenzocyclooctyne (DBCO), and
wherein the functionalized vesicle is a liposome covalently attached directly or via a linker to dibenzocyclooctyne (DBCO), and said liposome encapsulates an antibiotic.

27. The method of claim 16, wherein said therapeutic is vancomycin or daptomycin.

28. The method of claim 16, wherein said hydrogel is administered or implanted at a site of a prosthetic or surgical implant.

29. The method of claim 16, wherein said hydrogel is administered or implanted at a bone flap.

30. The method of claim 16, wherein said hydrogel is administered or implanted at a site of infection.

31. The method of claim 16, wherein the hydrogel is functionalized with an alkyne group.

32. The method of claim 31, wherein the hydrogel is functionalized with DBCO.
